(19) United States
(12) Patent Application Publication
(10) Pub. No.: US 2025/0041663 A1
(43) Pub. Date: Feb. 6, 2025

(54) DEVICE TRAINING AT A DETERMINED QUANTIZATION

(71) Applicant: Micron Technology, Inc., Boise, ID (US)

(72) Inventors: Shashank Bangalore Lakshman, Folsom, CA (US); Pavana Prakash, Houston, TX (US); Poorna Kale, Folsom, CA (US)

(21) Appl. No.: 18/753,758

(22) Filed: Jun. 25, 2024

Related U.S. Application Data
(60) Provisional application No. 63/517,825, filed on Aug. 4, 2023.

(51) Int. Cl. A63B 24/00; G06F 18/20; G06N 20/20

(52) U.S. Cl. CPC A63B 24/0062; G06F 18/20; G06N 20/20; A63B 2024/0065; A63B 2230/04; A63B 2230/207

(57) ABSTRACT

Systems, methods, devices, and apparatus are provided for device training at a determined quantization. For instance, a device can include a battery, a communication node, a sensor configured to collect user fitness data, and a processor coupled to the battery, the communication node, and the sensor. The processor can be configured to monitor a power state of the battery, determine a usage pattern of the electronic device and determine a quantization at which to train a machine learning model for analyzing the user fitness data based on the power state and the usage pattern. In addition, the processor can be configured to train the machine learning model at the determined quantization using the user fitness data and transmit the determined quantization and an update to the trained machine learning model via the communication node without transmitting the user fitness data.

### CROSS-REFERENCE TO RELATED APPLICATION

This application claims the benefit of U.S. Provisional Application No. 63/517,825, filed on Aug. 4, 2023, the contents of which are incorporated herein by reference.

### TECHNICAL FIELD

The present disclosure relates generally to apparatuses, systems, non-transitory machine-readable media, and methods associated with training devices at a determined quantization.

### BACKGROUND

A computing device can be, for example, a laptop computer, a desktop computer, a tablet, a smart phone, a mobile device, a digital camera, or wearable devices such as smart glasses, a wrist-worn device, and/or redundant combinations thereof, among other types of computing devices.

Computing devices can be used to implement machine learning models. Computing devices can also be used to train the machine learning models.

### BRIEF DESCRIPTION OF THE DRAWINGS

FIG. 1 illustrates example computing systems for training a machine learning model in accordance with some embodiments of the present disclosure.

FIG. 2 illustrates a block diagram for training a machine learning model in accordance with some embodiments of the present disclosure.

FIG. 3 is a flow diagram corresponding to a method for training a machine learning model in accordance with some embodiments of the present disclosure.

FIG. 4 is a flow diagram for training a machine learning model in accordance with some embodiments of the present disclosure.

FIG. 5 is a block diagram of an example computer system in which embodiments of the present disclosure may operate.

### DETAILED DESCRIPTION

Apparatuses, systems, machine-readable media, and methods related to training electronic devices at a determined quantization are described herein. Wearable electronic devices may enable state of the art Internet of Things (IoT) applications in fitness, health, and sports (FHS). Sensors on devices worn by a user may be used to monitor user state and behavior. For example, the sensors can gather and the devices can store user fitness data. Wearable devices come in a variety of form factors and specifications to cater to low, mid, and high-end market segments.

Machine learning applications can enable better guidance to users by analyzing user activity and/or performance and enable ways to achieve FHS goals. However, this could expose users to potential privacy issues relating to their personally identifiable information, as well as make them vulnerable to adversaries collecting such data for malicious uses, particularly if the user data is transmitted to another device and/or across a network.

One or more embodiments of the present disclosure address the above and other drawbacks of some approaches. For example, electronic devices can collect user fitness data, monitor a power state of a battery, and determine a usage pattern. A quantization at which to train a machine learning model for analyzing user fitness data can be determined based on the power state and the usage pattern. The electronic device can train the machine learning model at the determined quantization with the user fitness data. An indication of the determined quantization and an update to the machine learning model can be transmitted (e.g., to a central server) without transmitting the user fitness data.

In various instances, electronic devices can be utilized to implement federated learning. Federated learning describes the training of an algorithm using multiple decentralized electronic devices. In various examples, each of the electronic devices can receive an initial model. The electronic devices receive an initial model from a centralized server. The initial model can be utilized by the electronic devices for operation of the electronic devices until user fitness data is collected and a locally trained machine learning model is generated. The locally trained machine learning model (or updates to the initial model) can be transmitted to the central server, which can aggregate the updates or locally trained models received from multiple devices into a global updated federated model, which can be delivered back to the electronic devices for operation thereof.

A central server can share the initial model with multiple electronic devices. The processing resources and/or memory resources of an electronic device can train the initial model to create updates to the initial model and/or to generate a locally trained machine learning model. The electronic device can obtain user fitness data while the electronic device is in operation. The user fitness data can be used by the electronic device to train the received initial model to create the updates to the initial model and/or to generate the locally trained machine learning model. The electronic devices can perform training using their local computational resources independently of each other. Quantization data, updates to the initial model, and/or the locally trained machine learning model can be uploaded to the central server from the electronic devices. A locally trained machine learning model is distinguished from updates to the initial model in that the locally trained machine learning model refers to the entire model, whereas the model updates refers to any change to the initial model, such as updated weights, biases, etc.

The electronic devices determine a quantization at which to train the initial model. The quantization refers to how much time and resources of the electronic device are devoted to training the model (e.g., “how hard” the electronic device trains the model). The quantization can be determined by the electronic device based on a power state of a battery of the electronic device, a usage pattern of the electronic device, memory and/or processing capabilities of the electronic device, and/or other factors. For example, if the power state of the battery is relatively high, then the initial model can be trained at a greater quantization versus a relatively lower power state. As another example, if the usage pattern of the device suggests that usage will be relatively low in a coming period of time, then the initial model can be trained at a greater quantization versus a usage pattern that suggests relatively greater usage in the coming period of time. The electronic device can send an indication of the quantization (“quantization data”) at which it trained the initial model to the central server along with any update or trained machine learning model that it sends to the central server. The quantization data can be sent to the central server. The electronic devices can send updates to the initial model and/or quantization data without sending the user fitness data, thereby protecting any personally identifiable information that may be embedded in the user fitness data to protect the user's privacy.

The central server can collect respective model updates and quantization data from the multiple electronic devices. The central server can aggregate the received information to update the initial model to generate an updated model. The central server can use the quantization data to determine a respective weightage parameter to apply to the model updates received from each electronic device when aggregating the updates. For example, the central server can apply a greater weightage parameter to an update received from a first electronic device with an indication of a greater quantization than from a second electronic device with an indication of a lesser quantization so that the update from the first electronic device has a greater influence on the global update to the initial model. The indication of the relatively greater quantization can imply that the updates are more reliable than those associated with a relatively lesser quantization.

The central server can transmit the updated model for use by the electronic devices. The electronic devices can use the updated model for operation. In some embodiments, the electronic devices can further train the updated model and/or send additional updates to the central server.

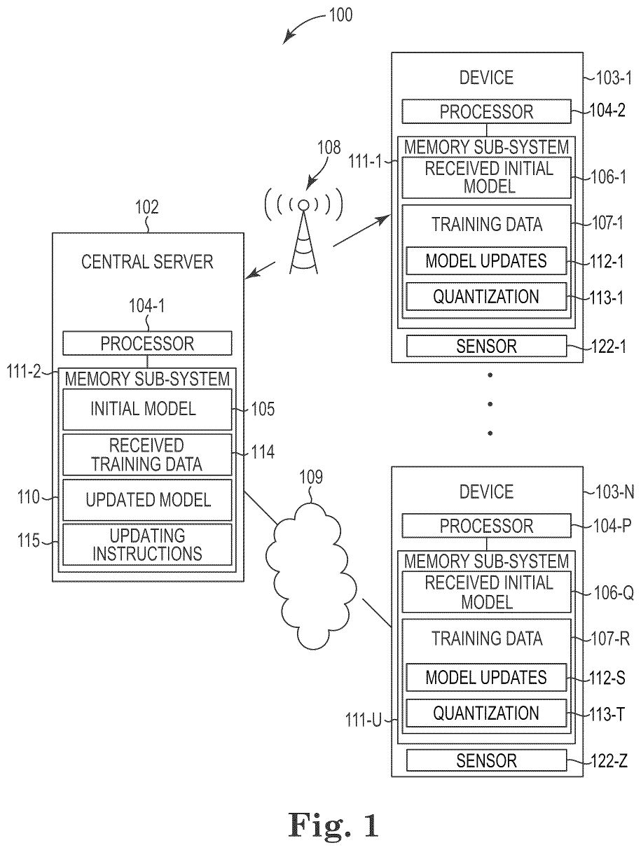

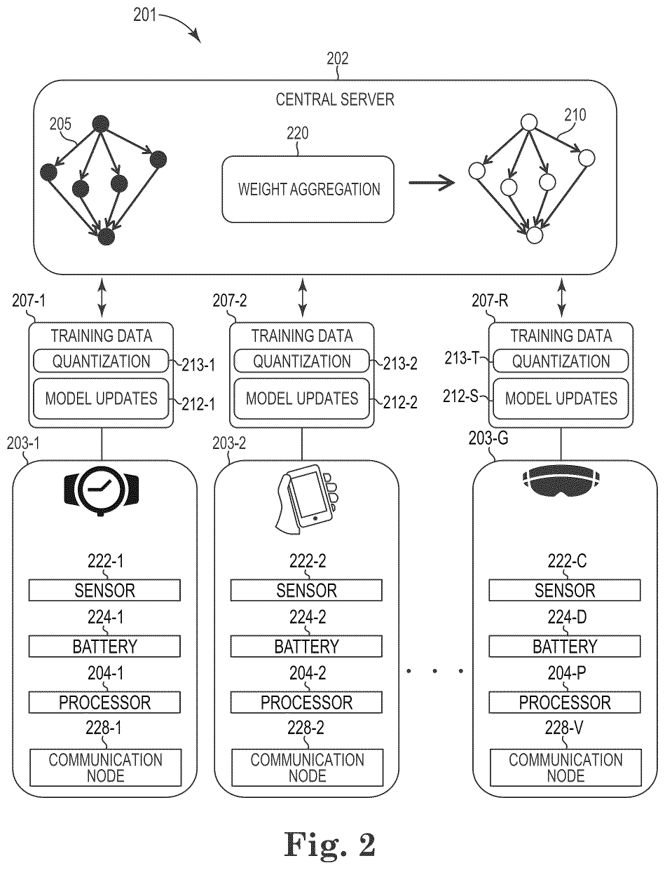

The figures herein follow a numbering convention in which the first digit or digits correspond to the figure number of the drawing and the remaining digits identify an element or component in the drawing. Similar elements or components between different figures may be identified by the use of similar digits. For example, reference numeral 102 may reference element “02” in FIG. 1, and a similar element may be referenced as 202 in FIG. 2. Multiple analogous elements within one figure may be referenced with a reference numeral followed by a hyphen and another numeral or a letter. For example, 203-1 may reference element 203-1 in FIGS. 2 and 203-2 may reference element 203-2, which may be analogous to element 203-1. Such analogous elements may be generally referenced without the hyphen and extra numeral or letter. For example, elements 203-1 and 203-2 or other analogous elements may be generally referenced as 203.

FIG. 1 illustrates example computing system 100 for training a machine learning model in accordance with some embodiments of the present disclosure. The computing system 100 can comprise a central server 102 and electronic devices 103-1, 103-N (e.g., devices 103-1, 103-N). The electronic devices 103 can include a processor coupled to a battery, communication node, and one or more sensors 122-1, 122-Z.

The computing system 100, the central server 102, and the electronic devices 103 can comprise hardware, firmware, and/or software configured to train a machine learning model. As used herein, a machine learning model can include a plurality of weights, biases, and/or activation functions among other variables that can be used on the electronic devices 103. The central server 102 and the electronic devices 103 can further include memory sub-systems 111-1, 111-2, 111-U (e.g., including a non-transitory machine readable medium (MRM)) on which may be stored instructions (e.g., updating instructions 115) and/or data (e.g., received training data 114, initial model 105, updated model 110, training data 107 including model updates 112 and/or quantization 113). Although the following descriptions refer to a processing device and a memory device, the descriptions may also apply to a system with multiple processing devices and multiple memory devices. In such examples, the instructions may be distributed across multiple memory devices for storage thereby and the instructions may be distributed across multiple processing devices for execution thereby.

The memory sub-systems 111 may include memory devices. The memory devices may be electronic, magnetic, optical, or other physical storage devices that store executable instructions and/or data. The memory devices may be, for example, non-volatile or volatile memory. In some examples, the memory devices can be a non-transitory MRM comprising random access memory (RAM), an Electrically-Erasable Programmable ROM (EEPROM), a storage drive, an optical disc, and the like. The memory sub-systems 111 may be disposed within a controller, the central server 102, and/or the electronic devices 103. In this example, the executable instructions (e.g., updating instructions 115) can be “installed” on the central server 102. The memory sub-systems 111 can be portable, external, or remote storage mediums, for example, that allow the central server 102 and/or the electronic devices 103 to store instructions and/or data. In this situation, the initial model 105 may be part of an “installation package” sent to the electronic devices 103 by the central server 102. The memory sub-system 111-2 of the central server 102 can be encoded with executable instructions for sending the initial model 105 to the electronic devices 103. In some examples, the processors 104 of the electronic devices 103 may be configured to receive the initial model 105 via a communication node.

The central server 102 can execute instructions using the processor 104-1 also referred to herein as processing device 104-1. The instructions can be stored in the memory sub-system 111-2 prior to being executed by the processing device 104-1. For example, the execution of the instructions can cause the initial model 105 to be provided to the electronic devices 103.

In some embodiments, the central server 102 can send the initial model 105 to each electronic device 103. The electronic device 103-1 can store the received initial model 106-1. The electronic device 103-N can store the received initial model 106-Q. The central server 102 can provide the initial model 105 to the electronic devices 103 utilizing a wireless network 108 and/or a physical network 109.

The electronic devices 103 can store the received initial model 106, user fitness data, and training data 107. The electronic devices 103, comprising the processors 104-2, 104-P (also referred to herein as processing device 104-2, 104-P), can execute the received initial model 106 utilizing the processors 104-2, 104-P to operate the electronic devices 103. The electronic device 103 can use user fitness data to train and update the received initial model 106. The initial model 105 can be trained using the user fitness data at a determined quantization 113, described in more detail below. An indication of the determined quantization is referred to herein as quantization data. The terms quantization and quantization data may be used interchangeably.

The model updates 112 are the result of updating the received initial model 106. For example, the electronic device 103 can use information based on the activity of a user while the user is wearing or in possession of the electronic device 103, referred to herein as user fitness data, to create the model updates 112. The sensor 122 in the electronic device 103 can monitor the behavior and state of the user to obtain information based on the activity of the user. Monitoring the behavior and state of the user can involve monitoring one or more of the heart rate, body temperature, oxygen saturation, etc. of the user. The electronic device 103 can use the information obtained from the activity of the user to train the received initial model 106, thereby creating the model updates 112. The model updates 112 do not include user fitness data. However, user fitness data (e.g., monitored heart rate, body temperature, oxygen saturation, etc.) may be used to create the model updates 112. The electronic device 103 can transmit the model updates 112 without transmitting personally identifiable information included in the user fitness data to the central server 102. The personally identifiable information of the user of the electronic device 103 remains secure and private when data is sent to the central server 102.

The electronic device 103 can determine a quantization at which to train the received initial model 106. In some embodiments, the electronic device 103 can store data comprising an indication of the quantization 113 with the training data 107. The quantization 113 can include information related to how often the received initial model 106 is trained, the amount of resources of the electronic device 103 that are used for the training, the total duration of the training, and/or other information that indicates how much training of the received initial model has occurred. For example, the quantization 113 may include the time of day that the received initial model 106 was trained and for how long, the amount of battery power the electronic device 103 had when the training occurred, the memory availability of the electronic device 103 when the training occurred, the processing power of the electronic device 103 when the training occurred, and/or the charging schedule of the electronic device 103, etc. Furthermore, in some embodiments, the electronic device 103 may be configured to collect the respective user fitness data in an amount dependent upon the power state of the electronic device 103. However, embodiments are not so limited. The electronic device 103 can collect data based on memory available on the electronic device 103, the processing power of the electronic device, and/or activity of the electronic devices 103, etc.

In some examples, the processor 104 can be configured to store quantization data 113. The processor 104 can be configured to monitor the power state of the battery, determine the usage pattern of the wearable electronic device 103, determine a quantization 113 at which to train the received initial model 106 for analyzing the user fitness data based on the power state and the usage pattern, train the received initial model 106 at the determined quantization 113 with the user fitness data, and/or transmit model updates 112 and the quantization data 113 via the communication node without transmitting the personally identifiable information of the user. In some embodiments, as described herein, the processor 104 can be configured to determine the quantization 113 based on a time of day, determine the quantization 113 based on a memory availability of the electronic device 103, determine the quantization 113 based on a time remaining until a next charge of the battery according to the usage pattern, and/or determine the quantization 113 based on environmental conditions.

The quantization 113 sent by each electronic device 103 allows the central server 102 to determine the level of importance to give the information provided in the model updates 112. For example, if the quantization 113 informs the central server 102 that the electronic device 103-1 has a higher processing power, as compared to another electronic device 103-N, the model updates 112-1 from the electronic device 103-1 may be given a higher importance than the model updates 112-S from another electronic device 103-N when creating the updated model 110. Similarly, if the quantization 113 informs the central server 102 that the user fitness data from the electronic device 103-1 was obtained with lower battery charge and the user fitness data from electronic device 103-N was obtained with a higher battery charge, the quantization 113-T from electronic device 103-N may be given a higher level of importance. The central server 102 can use training data 107 from all electronic devices 103 to create the updated model 110. The level of importance is also referred to herein as a respective weightage parameter that can be calculated for and applied by the central server 102 to information received from each of the electronic devices 103 when creating the updated model 110.

The central server 102 can receive an output from the electronic device 103. The output can be the training data 107 sent from the electronic device 103, which can be stored by the central server 102 as received training data 114. The training data 107 may be sent to the central server 102 utilizing a wireless network 108 and/or a physical network 109. Each of the electronic devices 103 can update the received initial model 106 to provide training data 107 for generating an updated model 110. The central server 102 can utilize the received training data 114 to generate corrections (e.g., training feedback) for the initial model 105. The corrections can be used to modify the weights, and/or biases of the initial model 105 to create the updated model 110. The corrections can be provided to the electronic devices 103 (not shown). For instance, when an updated model 110 is created by the central server 102, updated instructions are also created to determine when the updated model will be sent to the electronic devices 103. As used herein, the updated model 110 can describe an initial model that has been updated or trained using aggregated received training data 114.

The central server 102 can execute updating instructions 115 using the processing device 104-1 to create the updated model 110. The updating instructions 115 can be stored in the memory sub-system 111-2 prior to being executed by the processing device 104-1. The execution of the updating instructions 115 can cause the received training data 114, specifically the model updates 112, to be aggregated into the initial model 105, thereby creating the updated model 110. Each of the model updates 112 can be aggregated into the initial model 105 according to the quantization associated therewith. The updated model 110 can be sent to the electronic devices 103. The updated model 110 is also referred to herein as a global updated model. The updating instructions 115 can be software, encoded in a computer readable-medium, or can be logic.

Once the updated model 110 is received by the electronic devices 103, the electronic devices 103 may be configured to operate based on the updated model 110. In some embodiments, the electronic devices 103 may continue to train the updated model 110. For example, when the updated model 110 is sent to the electronic device 103-1, the sensor 122-1 of the electronic device 103-1 may continue to gather data related to the use of the electronic device 103-1. The additional data gathered may then be used to create additional updates to the updated model 110. Further, a different quantization at which to train the updated model 110 may be determined as additional data is gathered. The electronic device 103-1 can collect additional user fitness data and train the updated model 110 with the additional user fitness data at the newly determined quantization. The additional model updates and the additional quantization data may be combined to create additional training data to be sent to the central server 102 to produce an additional updated model.

In various examples, the processors 104 can be internal to the memory sub-systems 111 instead of being external to the memory sub-systems 111 as shown. For instance, the processors 104 can be processor in memory (PIM) processors. The processors 104 can be incorporated into the sensing circuitry of the memory sub-systems 111 and/or can be implemented in the periphery of the memory sub-system 111, for instance. The processors 104 can be implemented under one or more memory arrays of the memory sub-system 111.

FIG. 2 illustrates a block diagram 201 for training a machine learning model in accordance with some embodiments of the present disclosure. FIG. 2 includes a central server 202 and a plurality of electronic devices 203-1, 203-2, 203-G. The central server 202 can store an initial model 205 and an updated model 210. The central server 202 can store instructions for performing weight aggregation to create the updated model 210 using training data 207 received from electronic devices 203. In various instances, the instructions can be implemented as software, hardware, and/or firmware. The instructions can be executed by processor 104-1 of FIG. 1.

The electronic devices 203 can be a plurality of wearable and/or portable devices. For example, the electronic devices 203 can be tablets, cellular phones, mobile computing devices, and virtual reality (VR) headsets, smart watches, etc. The electronic devices 203 illustrated in FIG. 2 include a smart watch 203-1, cellular phone 203-2, and virtual reality (VR) headset 203-G. However, the electronic devices 203 can comprise different types of electronic devices other than those shown. Each electronic device 203 includes a respective sensor 222-1, 222-2, 222-C, a respective battery 224-1, 224-2, 224-D, a respective processor 204-1, 204-2, 204-P, and a respective communication node 228-1, 228-2, 228-V, among other components not specifically illustrated.

The central server 202 can transmit an initial model 205 for analyzing user data (e.g., user fitness data). The central server 202 can provide the initial model 205 by sending the initial model 205 to the electronic devices 203. For instance, the central server 202 can send the initial model 205 to the first electronic device 203-1, the second electronic device 203-2, and the third electronic device 203-G. The initial model 205 can comprise weights, biases, and/or functions for the electronic device 203 to use when performing daily functions, such as analyzing user fitness data. As described herein, each electronic device 203 can be configured to receive the initial model 205, collect respective user fitness data, determine a respective quantization 213 at which to train the initial model 205, and transmit the respective quantization data 213 and model updates 212 to the central server 202 without transmitting user fitness data, which may include respective personally identifiable information of the users.

While the electronic device 203 is being used by the user, the sensor 222 of the electronic device 203 can gather data (e.g., fitness data) related to the use of the electronic device 203 and the state of the user. For example, the electronic device 203 can gather information related to the inertial measurement unit (IMU), heart rate, body temperature, oxygen saturation, stress levels, electrocardiogram (ECG), etc. Each electronic device 203 can use the data (e.g., fitness data) gathered by the respective sensor 222 to train the initial model 205 received from the central server 202. Fitness data may be gathered, by the sensor 222, as the user wears and/or is in possession of the electronic device 203 and performs everyday activities, such as relaxing, working, exercising, sleeping, etc. The data obtained by the sensor 222 can be used to create model updates 212 that can be used by the electronic device 203 when performing daily functions. For example, the sensor 222 can be a heart rate sensor to detect the heart rate of the user throughout the day as the user uses the electronic device 203. The electronic device can use the data collected by the heart rate sensor to train the initial model 205 and create the model updates 212.

The model updates 212 can be a compilation of updates to the initial model 205 based on user specific data. However, the model updates 212 do not include personally identifiable information of the user. The electronic device 203 can make the updates to the initial model 205 using the user fitness data without incorporating the personally identifiable information of the user fitness data into the model itself or transmitting the personally identifiable information of the user fitness data with the updated model.

The training data 207 can also include quantization data 213. In some embodiments, in addition to data describing the circumstances around training the initial model, the quantization data 213 can describe circumstances surrounding the collection of user fitness data. The quantization data 213 can describe the environmental conditions, the battery strength, the time of day, the strength of the processor 204 in the electronic device 203, etc. These factors can assist in determining the accuracy of the trained machine learning model 212 and the training data 207. For example, an electronic device 203-2 with a stronger processing device 204-2, than another electronic device 203-G, may create a more reliable model updates 212-2 than the electronic device 203-G with the weaker processing device 204-P. For instance, a processor 204-2 with stronger processing power would provide a more reliable model updates 212-2 than a processor 204-P with weaker processing power.

In another example, a heart rate monitor sensor may provide better tracking of the heart rate with a full battery charge rather than a low battery charge. In this example, the quantization data 213 may include the battery condition in which the information was gathered. In addition, the quantization data 213 can include the weather conditions surrounding the collection of data. For instance, if the electronic device 203 is collecting user fitness data related to oxygen saturation, the quantization data 213 including information of the weather (e.g., sunny day, rainy day, etc.) could determine the accuracy and/or reliability of the data collected. Similarly, the quantization data 213 including information related to the activity of the user (e.g., running, walking, sitting, etc.), while the data is collected could determine the level of importance of the model updates 212.

In some embodiments, the processor 204 will cause the communication node 228 to send, via a wireless network (e.g., wireless network 108 of FIG. 1) and/or a physical network (e.g., physical network 109 of FIG. 1), the training data 207, including the quantization data 213 and the model updates 212 without the user fitness data and/or personally identifiable information to the central server 202. In some embodiments, the electronic device 203 may transmit an indication of the quantization 213 based on the determined amount of memory available, the determined power state, and/or the determined processing power. The central server 202 can perform weight aggregation 220 on training data 207 received from the electronic devices 203. For instance, quantization data 213 from each electronic device 203 can be received by the central server 202. The central server 202 can analyze the quantization data 213 received from each electronic device 203 to determine the accuracy, reliability, and/or importance of the user fitness data without the personally identifiable information of the user of each electronic device 203. The central server 202 can then aggregate the respective received model updates 212 based on the quantization data 213 associated therewith to generate an updated model 210.

Aggregating the received data can include processing multiple sets of training data 207 received from different electronic devices 203. For instance, the training data 207-1 provided by electronic device 203-1 can be a first set of training data 207-1 (including quantization data 213-1 and model updates 212-1), the training data 207-2 (including quantization data 213-2 and model updates 212-2) provided by electronic device 203-2 can be a second set of training data 207-2, and the training data 207-R (including quantization data 213-T and model updates 212-S) provided by electronic device 203-G can be a third set of training data 207-R. The central server 202 can process the first set of training data 207-1, the second set of training data 207-2, and the third set of training data 207-R to generate a single updated model 210. Once the updated model 210 is generated, the central server 202 can send the updated model 210 to the electronic devices 203 utilizing a wireless network (e.g., wireless network 108 of FIG. 1) and/or a physical network (e.g., physical network 109 of FIG. 1).

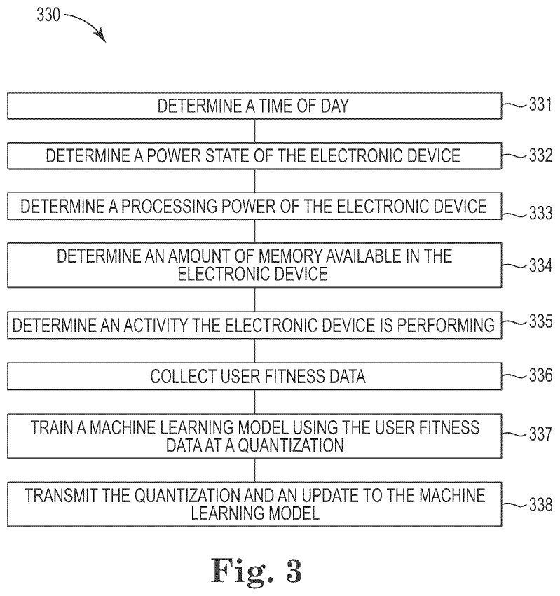

FIG. 3 is a flow diagram corresponding to a method 330 for training a machine learning model in accordance with some embodiments of the present disclosure. The method 330 may be performed, in some examples, using a computing system such as those described with respect to FIGS. 1 and 2. At 331, the time of day in which the electronic device is being used can be determined. At 332, a power state of an electronic device can be determined. In some embodiments, the electronic device may determine a quantization at which to train an initial machine learning model. The determined quantization can be represented by quantization data, which can indicate the accuracy, reliability, and/or importance of updates to the initial model created by the electronic device as a result of training the initial model based on user fitness data collected from a sensor. For example, the amount of battery life an electronic device has can indicate the amount of power the processor has to train the model. The time of day in which training is performed can also be indicative of the accuracy of the training. For example, if the training is performed at a time when the electronic device is typically busy performing other tasks, the accuracy of the training could be hindered. The central server can use the quantization data, including information related to the power state of the electronic device when the training occurred, to determine the importance of the model updates, which do not include personally identifiable information of the user, when generating an updated model. The central server can use the quantization data to determine how updates to the model are aggregated with other updates by other electronic devices into a global updated model.

At 333, a processing power of the electronic device can be determined. At 334, an amount of memory available in the electronic device can be determined. In some embodiments, the quantization data can indicate the processing power of the processing device in the electronic device and the state of the memory in the electronic device. The ability of the processing device in the electronic device can determine the accuracy of the accuracy of the training of the initial model performed by the electronic device. An electronic device with a high-powered processing device can provide more accurate results than an electronic device with a low-powered processing device. The state of the memory can refer to a total amount of memory, an amount of free memory, an age or wear status of the memory, etc. The state of the memory of the electronic device can be indicative of the completeness of the gathered data. For example, a sensor on an electronic device with a low memory may save memory space by only storing a portion of the data collected. Using only a portion of the data collected to generate model updates could reduce their accuracy.

In some embodiments, when quantization data is sent to a central server, the central server can use the quantization data to create an accurate and reliable updated model. The central server is able to prioritize each training data received when aggregating the model updates received from the plurality of electronic devices. For instance, the central server is further configured to compute a respective weightage parameter for each respective model update based on each respective quantization data, and aggregate the respective model updates based on the respective weightage parameters.

At 335, an activity the electronic device is performing can be determined. In some embodiments, the activity being performed, the environmental factors, and the state of the electronic device can be used to create quantization data. The level of importance of the data collected can vary depending on the activity the electronic device is performing. For example, heart rate information obtained while a user is running (e.g., the electronic device being in exercise mode) may have a higher importance than heart rate information obtained while a user is resting. Hence, the quantization data can use information such as the activity of the electronic device to provide information related to the level of importance of the training data when provided to the central server. The quantization data can provide information related to the importance of the activity used to generate the user fitness data without providing the user fitness data itself and/or personally identifiable information.

At 336, user fitness data can be collected by the electronic device. In some embodiments, the user fitness data can be used to train an initial machine learning model. The electronic device can use sensors to gather user fitness data based on the use of the electronic device by the user.

At 337, an initial model can be trained using the user fitness data at a quantization. The training can result in model updates. In some embodiments, the electronic device can use machine learning to generate improvements to an initial model provided by the central server. For instance, as the user uses the electronic device and/or provides input to the electronic device, the electronic device can learn user preferences and other, more efficient ways to perform functions.

In some embodiments, the electronic device can learn more efficient ways to perform functions by analyzing the information collected by the sensors and the activities of the user and electronic device. For example, the results of a sensor collecting oxygen saturation data of a user in rainy weather versus in sunny weather or while the user is running versus while the user is walking. Analyzing the difference in each situation may lead to a more efficient way for the electronic device to perform functions. In addition, the user can make selections related to data collected by the sensor. The selections made by the user can be used by the electronic device to provide training to the electronic device and develop a trained machine learning model.

At 338, the quantization and the model updates to the initial machine learning model can be transmitted. The training data may be transmitted to the central server via a communications node located on the electronic device without transmitting the user fitness data. The central server may aggregate model updates received from multiple electronic devices to develop updates to the initial model, creating an updated model.

The quantization data can include information surrounding the circumstances on how the training of the initial machine learning model occurred. Examples include the environmental conditions when the training occurred, the battery conditions of the electronic device when the training occurred, the charging cycle of the electronic device when the training occurred, the processing power and/or memory levels of the electronic device when the training occurred, the time of day when the training occurred, and/or activity level of the user as the data was collected. The quantization data can assist the central server in determining how to combine the training data received from a plurality of electronic devices. Based on the quantization data, the central server will determine the importance, accuracy, reliability, and/or relevance of the model updates provided by the plurality of electronic devices in order to aggregate the individual updates into the global updated model. The global updated model may then be sent to the electronic devices. The electronic devices may operate based on the global updated model received from the central server. In some examples, the electronic device may provide suggestions to the user based on the updated model.

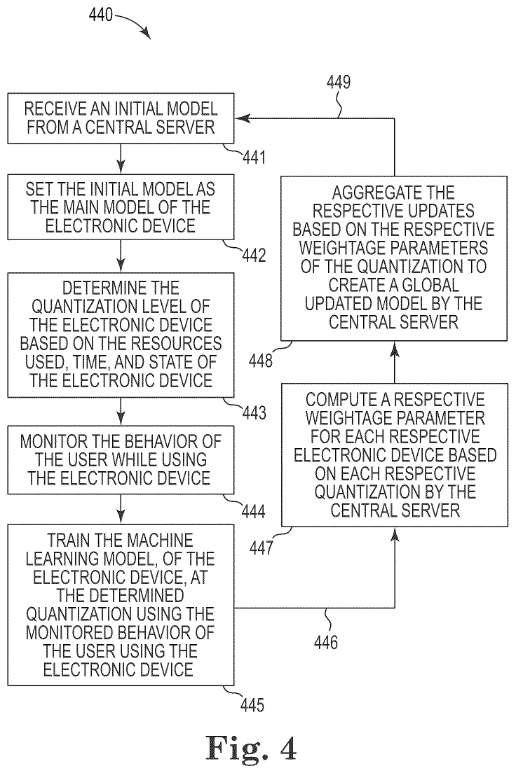

FIG. 4 is a flow diagram 440 for training a machine learning model in accordance with some embodiments of the present disclosure. FIG. 4 describes an example of using user fitness data, while omitting personally identifiable information, and quantization data (e.g., quantization 113 of FIG. 1) about the electronic devices (e.g., the electronic device 103 of FIG. 1) to update an initial model (e.g., initial model 105 of FIG. 1) provided by a central server (e.g., the central server 102 of FIG. 1). At 441 the flow diagram 440 describes the electronic device receiving an initial model (e.g., received initial model 106 of FIG. 1) from the central server. At 442 the flow diagram 440 describes the electronic device setting the initial model as the main model. The central server can send an initial model to an electronic device or a plurality of electronic devices. In some embodiments, the type of initial model sent to the electronic device may depend on the type of electronic device. The processor (e.g., processor 104 of FIG. 1) of the electronic device can use the initial model for initial operation of the electronic device. The electronic device can use the initial model until the electronic device has had a chance to collect user fitness data.

At 443 the flow diagram 440 describes determining the quantization level of the electronic device based on the resources used, time, and state of the electronic device. The electronic device can use the resources (e.g., processing power, etc.), time of data collection, state of the electronic device, as well as other quantization data, to determine the quantization at which to train the initial model. At 444 the flow diagram 440 describes monitoring the behavior of the user while using the electronic device. The behavior of the user may be monitored to produce user fitness data. The quantization level of the electronic device may be determined prior to collecting user fitness data, during the collection of user fitness data, and/or immediately after collecting user fitness data. The quantization level can provide perspective on how to apply the user fitness data without providing personally identifiable information.

At 445 the flow diagram 440 describes training the machine learning model of the electronic device at the determined quantization using the monitored behavior of the user. The machine learning model can be trained using user fitness data. For example, information from user activity can be used to train the machine learning model. However, the trained machine learning model will not include personally identifiable information that may be contained in the user fitness data. The electronic device can then send, at 446, the quantization data collected by the electronic device to the central server along with the model updates (e.g., model updates 212 of FIG. 2). At 447 the flow diagram 440 describes computing a respective weightage parameter for each respective electronic device based on each respective quantization by the central server. At 448 the flow diagram 440 describes aggregating the respective updates based on the respective weightage parameters of the quantization to create a global updated model (e.g., updated model 110 of FIG. 1) by the central server. The central server uses the quantization data received from each electronic device to determine the importance of the information collected when creating a global updated model for the electronic devices.

The central server will then send, at 449, the global updated model to the electronic devices connected to the central server. In some embodiments, the global updated model sent to the electronic devices may be based on the type of electronic device receiving the global updated model. For example, as described herein, the electronic devices can receive the same global updated model or a global updated model that is tailored to the type of electronic device receiving the global updated model. In some embodiments, the electronic device can set the global updated model as the base operations and train the machine learning model based on the global updated model.

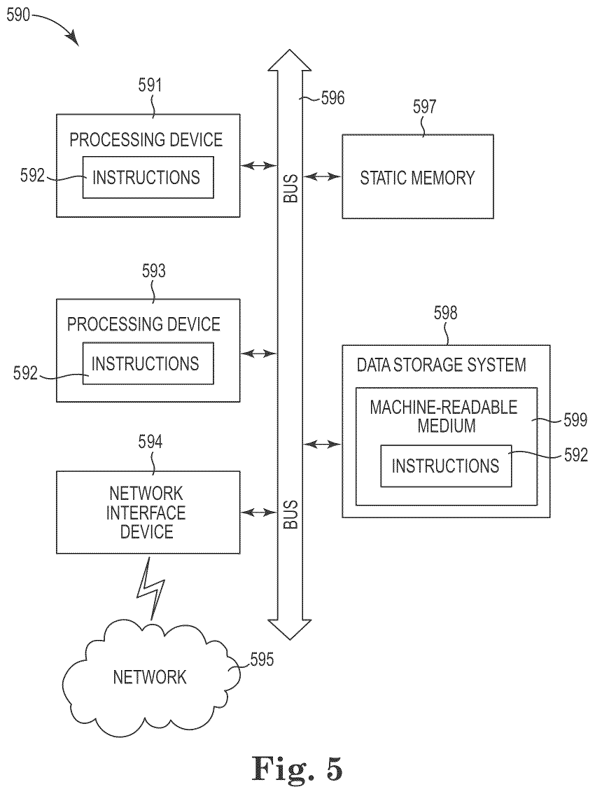

FIG. 5 is a block diagram of an example computer system 590 in which embodiments of the present disclosure may operate. For example, FIG. 5 illustrates an example machine of a computer system 590 within which a set of instructions, for causing the machine to perform any one or more of the methodologies discussed herein, can be executed. In some embodiments, the computer system 590 can correspond to a host system that includes, is coupled to, or utilizes a memory sub-system (e.g., the memory sub-systems 111-1, 111-2, 111-U of FIG. 1). The computer system 590 can be used to perform the operations described herein (e.g., to perform operations corresponding to the processors 104-1, 104-2, 104-P of FIG. 1). In alternative embodiments, the machine can be connected (e.g., networked) to other machines in a LAN, an intranet, an extranet, the Internet, and/or wireless network. The machine can operate in the capacity of a server or a client machine in client-server network environment, as a peer machine in a peer-to-peer (or distributed) network environment, or as a server or a client machine in a cloud computing infrastructure or environment.

The machine can be a personal computer (PC), a tablet PC, a set-top box (STB), a Personal Digital Assistant (PDA), a cellular telephone, a web appliance, a server, a network router, a switch or bridge, or any machine capable of executing a set of instructions (sequential or otherwise) that specify actions to be taken by that machine. Further, while a single machine is illustrated, the term “machine” shall also be taken to include any collection of machines that individually or jointly execute a set (or multiple sets) of instructions to perform any one or more of the methodologies discussed herein.

The example computer system 590 includes a processing device (e.g., processor) 591, a main memory 593 (e.g., read-only memory (ROM), flash memory, dynamic random access memory (DRAM) such as synchronous DRAM (SDRAM) or Rambus DRAM (RDRAM), etc.), a static memory 597 (e.g., flash memory, static random access memory (SRAM), etc.), and a data storage system 598, which communicate with each other via a bus 596.

The processing device 591 represents one or more general-purpose processing devices such as a microprocessor, a central processing unit, or the like. More particularly, the processing device 591 can be a complex instruction set computing (CISC) microprocessor, reduced instruction set computing (RISC) microprocessor, very long instruction word (VLIW) microprocessor, or a processor implementing other instruction sets, or processors implementing a combination of instruction sets. The processing device 591 can also be one or more special-purpose processing devices such as an application specific integrated circuit (ASIC), a field programmable gate array (FPGA), a digital signal processor (DSP), network processor, or the like. The processing device 591 is configured to execute instructions 592 for performing the operations and steps discussed herein. The computer system 590 can further include a network interface device 594 to communicate over the network 595.

The data storage system 598 can include a machine-readable storage medium 599 (also known as a computer-readable medium) on which is stored one or more sets of instructions 592 or software embodying any one or more of the methodologies or functions described herein. The instructions 592 can also reside, completely or at least partially, within the main memory 593 and/or within the processing device 591 during execution thereof by the computer system 590, the main memory 593 and the processing device 591 also constituting machine-readable storage media. The machine-readable storage medium 599, data storage system 598, and/or main memory 593 can correspond to the memory sub-systems 111-1, 111-2, 111-U of FIG. 1.

In one embodiment, the instructions 592 include instructions to implement functionality corresponding to training a machine learning model at a determined quantization (e.g., using processors 104-1, 104-2, 104-P of FIG. 1). While the machine-readable storage medium 599 is shown in an example embodiment to be a single medium, the term “machine-readable storage medium” should be taken to include a single medium or multiple media that store the one or more sets of instructions. The term “machine-readable storage medium” shall also be taken to include any medium that is capable of storing or encoding a set of instructions for execution by the machine and that cause the machine to perform any one or more of the methodologies of the present disclosure. The term “machine-readable storage medium” shall accordingly be taken to include, but not be limited to, solid-state memories, optical media, and magnetic media.

Some portions of the preceding detailed descriptions have been presented in terms of algorithms and symbolic representations of operations on data bits within a computer memory. These algorithmic descriptions and representations are the ways used by those skilled in the data processing arts to most effectively convey the substance of their work to others skilled in the art. An algorithm is here, and generally, conceived to be a self-consistent sequence of operations leading to a desired result. The operations are those requiring physical manipulations of physical quantities. Usually, though not necessarily, these quantities take the form of electrical or magnetic signals capable of being stored, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. The present disclosure can refer to the action and processes of a computer system, or similar electronic computing device, that manipulates and transforms data represented as physical (electronic) quantities within the computer system's registers and memories into other data similarly represented as physical quantities within the computer system memories or registers or other such information storage systems.

The present disclosure also relates to an apparatus for performing the operations herein. This apparatus can be specially constructed for the intended purposes, or it can include a general purpose computer selectively activated or reconfigured by a computer program stored in the computer. Such a computer program can be stored in a computer readable storage medium, such as, but not limited to, any type of disk including floppy disks, optical disks, CD-ROMs, and magnetic-optical disks, read-only memories (ROMs), random access memories (RAMs), EPROMS, EEPROMs, magnetic or optical cards, or any type of media suitable for storing electronic instructions, each coupled to a computer system bus.

The algorithms and displays presented herein are not inherently related to any particular computer or other apparatus. Various general purpose systems can be used with programs in accordance with the teachings herein, or it can prove convenient to construct a more specialized apparatus to perform the method. The structure for a variety of these systems will appear as set forth in the description below. In addition, the present disclosure is not described with reference to any particular programming language. It will be appreciated that a variety of programming languages can be used to implement the teachings of the disclosure as described herein.

The present disclosure can be provided as a computer program product, or software, that can include a machine-readable medium having stored thereon instructions, which can be used to program a computer system (or other electronic devices) to perform a process according to the present disclosure. A machine-readable medium includes any mechanism for storing information in a form readable by a machine (e.g., a computer). In some embodiments, a machine-readable (e.g., computer-readable) medium includes a machine (e.g., a computer) readable storage medium such as a read only memory (“ROM”), random access memory (“RAM”), magnetic disk storage media, optical storage media, flash memory devices, etc.

In the foregoing specification, embodiments of the disclosure have been described with reference to specific example embodiments thereof. It will be evident that various modifications can be made thereto without departing from the broader spirit and scope of embodiments of the disclosure as set forth in the following claims. The specification and drawings are, accordingly, to be regarded in an illustrative sense rather than a restrictive sense.

What is claimed is:

1. An electronic device, comprising:
a battery;
a communication node;
a sensor configured to collect user fitness data; and
a processor coupled to the battery, the communication node, and the sensor, wherein the processor is configured to:
monitor a power state of the battery;
determine a usage pattern of the electronic device;
determine a quantization at which to train a machine learning model for analyzing the user fitness data based on the power state and the usage pattern; and
transmit the determined quantization and an update to the machine learning model via the communication node without transmitting the user fitness data.

2. The electronic device of claim 1, wherein the processor is further configured to determine the quantization based on a time of day.

3. The electronic device of claim 2, wherein the processor is further configured to determine the quantization based on a memory availability of the electronic device.

4. The electronic device of claim 3, wherein the processor is further configured to determine the quantization based on a time remaining until a next charge of the battery according to the usage pattern.

5. The electronic device of claim 4, wherein the processor is further configured to determine the quantization based on environmental conditions.

6. The electronic device of claim 1, wherein the user fitness data includes one or more of heart rate, body temperature, and oxygen saturation.

7. The electronic device of claim 1, wherein the processor is configured to:
receive the machine learning model via the communication node as an initial model; and
receive an updated model via the communication node after transmitting the determined quantization and the update to the machine learning model.

8. The electronic device of claim 7, wherein the processor is further configured to provide suggestions to the user based on the updated model and based on the user fitness data.

9. A system, comprising:
a server configured to transmit an initial model for analyzing user fitness data; and
a plurality of electronic devices, each having different hardware specifications and each configured to:
receive the initial model;
collect respective user fitness data;
determine a respective quantization at which to train the initial model; and
transmit the respective quantization and a respective update to the initial model to the server without transmitting the respective user fitness data;
wherein the server is further configured to:
aggregate the respective updates based on the respective quantizations to create an updated model; and
transmit the updated model to the plurality of electronic devices.

10. The system of claim 9, wherein the server is further configured to compute a respective weightage parameter for each respective trained machine learning model based on each respective quantization; and
aggregate the respective updates based on the respective weightage parameters.

11. The system of claim 9, wherein the plurality of electronic devices are each configured to operate based on the updated model.

12. The system of claim 9, wherein the plurality of electronic devices are each configured to:
collect additional respective user fitness data; and
train the updated model at the determined quantization with the additional respective user fitness data.

13. The system of claim 9, wherein each of the plurality of electronic devices is configured to omit personally identifiable information of the user when transmitting data to the server.

14. The system of claim 9, wherein each of the plurality of electronic devices is configured to collect the respective user fitness data in an amount dependent upon a power state of the electronic device.

15. A method, comprising:
determining a time of day;
determining a power state of an electronic device;
determining a processing power of the electronic device;
determining an amount of memory available in the electronic device;
determining an activity the electronic device is performing;
collecting, by the electronic device, user fitness data;
training a machine learning model using the user fitness data at a quantization based on:
the time of day;
the amount of memory available;
the power state; and
the processing power; and
transmitting the quantization and an update to the machine learning model.

16. The method of claim 15, wherein transmitting the update to the machine learning model comprises transmitting an updated weight.

17. The method of claim 15, further comprising receiving a global updated model based on an aggregation of updates by a plurality of electronic devices.

18. The method of claim 17, further comprising operating the electronic device based on the global updated model.

19. The method of claim 17, further comprising providing suggestions to a user based on the global updated model.

20. The method of claim 17, comprising transmitting the quantization and the update without transmitting the user fitness data.
